(19) United States
(12) Patent Application Publication
(10) Pub. No.: US 2009/0023933 A1
(43) Pub. Date: Jan. 22, 2009

(54) METHOD FOR ISOLATING ACIDS FROM CHEMICAL REACTION MIXTURES BY USING 1-ALKYLIMIDAZOLES

(75) Inventors: Matthias Maase, Speyer (DE); Oliver Huttenloch, Neulussheim (DE)

Correspondence Address: CONNOLLY BOVE LODGE & HUTZ LLP, 1875 EYE STREET, N.W., SUITE 1100, WASHINGTON, DC, 20006, US

(73) Assignee: BASF Aktiengesellschaft, Ludwigshafen (DE)

(21) Appl. No.: 10/596,548

(22) PCT Filed: Dec. 17, 2004
(86) PCT No.: PCT/EP2004/014386

§ 371 Date: Jun. 16, 2006

(30) Foreign Application Priority Data
Dec. 19, 2003 (DE) 10360397.2

(51) Int. Cl. C07D 233/54

(52) U.S. Cl. 548/335.1

(57) ABSTRACT

Method of separating acids from reaction mixtures by means of an auxiliary base, where the auxiliary base
b) reacts with the acid to form a salt which is liquid at temperatures at which the desired product is not significantly decomposed while the liquid salt is being separated off and
c) the salt of the auxiliary base forms two immiscible liquid phases with the desired product or the solution of the desired product in a suitable solvent,
   in which the auxiliary base used is an alkylmidazole,
   which has a solubility in 30% strength by weight sodium chloride solution at 25° C. of 10% by weight or less and
   whose hydrochloride has a melting point below 55° C.

The present invention describes a method of separating acids from reaction mixtures in a simplified way by means of an ionic liquid based on 1-alkylimidazoles.

A practitioner of chemistry often has the problem of neutralizing acids liberated during a chemical reaction or separating acids from reaction mixtures. Examples of reactions in which acids are liberated during the course of the reaction are the silylation of alcohols or amines by halosilanes, the phosphorylation of amines or alcohols by phosphorus halides, the formation of sulfonic esters or sulfonamides from alcohols or amines and sulfonyl chlorides or sulfonic anhydrides, eliminations or substitutions.

These reactions liberate acids, for which reason an auxiliary base which generally does not participate as reactant in the actual reaction is additionally added. In general, it is necessary to bind the liberated acids by means of this base with formation of salts in order to suppress secondary and subsequent reactions or else simply in order to remove the acid from the desired reaction product and, if appropriate, return it to the process. If the salts of the bases used are not separated off initially, they can also be worked up in the presence of the desired product, e.g. by addition of a further, stronger base such as an aqueous caustic alkali, e.g. sodium hydroxide or potassium hydroxide solution. This forms the salt of the stronger base added in this step. In addition, the base originally used is liberated. These two components, i.e. the salt of the stronger base and the liberated base used initially (auxiliary base) as a rule likewise have to be separated off from the desired product. In this procedure, it is often disadvantageous that the desired product which is present in the work-up can be decomposed by the added stronger base itself or further substances in this base, e.g. the water in an aqueous caustic alkali.

The salts of the auxiliary base with the acid are generally insoluble in organic solvents and have high melting points, so that in organic media they form suspensions which are more difficult to handle than, for example, liquids. It would therefore be desirable to be able to separate off the salts of the auxiliary bases in liquid form. In addition, the known process engineering disadvantages of suspensions would be eliminated. These are, for example, the formation of encrustations, reduction of heat transfer, poor mixing and stirrability and also formation of local excess and deficient concentrations and hot spots.

Accordingly, the prior art has the following disadvantages for processes carried out industrially:
1) addition of two auxiliaries, namely the auxiliary base and a further strong base, and the resulting task of separating two auxiliaries from the desired product and from one another,
2) handling of suspensions,
3) removal of the salt of the strong base as a solid.

However, a phase separation which is simple in process engineering terms by means of a liquid-liquid phase separation is desirable.

WO 03/62171 discloses a method of separating acids from reaction mixtures by means of ionic liquids, in which lists of possible ionic liquids are given. However, some of the auxiliary bases listed there have quite high melting points, which means thermal stress on the desired product, and can be recovered only with losses of starting material because of their sometimes relatively high solubility in water. Owing to their solubility in water, they have to be recovered from water in a complicated distillation with an appropriately large number of theoretical plates or by means of liquid-liquid extraction.

It was an object of the present invention to develop a process for separating acids from reaction mixtures by means of ionic liquids which have low melting points and can easily be recovered.

This object is achieved according to the invention by a method of separating acids from reaction mixtures by means of an auxiliary base, where the auxiliary base
b) reacts with the acid to form a salt which is liquid at temperatures at which the desired product is not significantly decomposed while the liquid salt is being separated off and
c) the salt of the auxiliary base forms two immiscible liquid phases with the desired product or the solution of the desired product in a suitable solvent,
   in which the auxiliary base used is an alkylimidazole which has a solubility in 30% strength by weight sodium chloride solution at 25° C. of 10% by weight or less and whose hydrochloride has a melting point below 55° C.

In a preferred embodiment of the invention, the solubility of the free alkylimidazole in 30% strength by weight sodium chloride solution at 25° C. is 5% by weight or less, particularly preferably 3% by weight or less, very particularly preferably 1% by weight or less and in particular 0.5% by weight or less.

Here, 30% strength by weight sodium chloride solution serves as a standardized model system for determining the solubility of the 1-alkylimidazoles which are suitable for the purposes of the invention in aqueous systems. To carry out the method of the invention, a very low solubility in aqueous systems is important.

In a further, preferred embodiment, the melting point of the hydrochloride of the 1-alkylimidazoles which are suitable for the purposes of the invention is 50° C. or less, particularly preferably 45° C. or less, very particularly preferably 40° C. or less, in particular 35° C. or less and especially 30° C. or less.

Preferred alkylimidazoles which fulfill these conditions are alkylimidazoles of the formula (I),

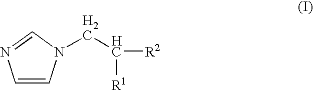

where R¹ and R² can each be, independently of one another, hydrogen or linear or branched C₁-C₆-alkyl, with the proviso that R¹ and R² have a total of at least 1 carbon atom and a total of not more than 6 carbon atoms, preferably have total of from 1 to 4 carbon atoms, particularly preferably have a total of 1 or 2 carbon atoms and very particularly preferably have a total of 2 carbon atoms.

Examples of R¹ and R² are hydrogen, methyl, ethyl, isopropyl, n-propyl, n-butyl, isobutyl, sec-butyl, tert-butyl and n-hexyl. Preferred radicals R¹ and R² are hydrogen, methyl and ethyl.

Examples of compounds of the formula (I) are n-propylimidazole, n-butylimidazole, isobutylimidazole, 2′-methylbutylimidazole, isopentylimidazole, n-pentylimidazole, isohexylimidazole, n-hexylimidazole, isooctylimidazole and n-octylimidazole.

Preferred compounds (I) are n-propylimidazole, n-butylimidazole and isobutylimidazole, with particular preference being given to n-butylimidazole and isobutylimidazole and very particular preference being given to n-butylimidazole.

According to the invention, the auxiliary base used can be one of the abovementioned compounds which
b) reacts with the acid eliminated during the reaction to form a salt which is liquid at temperatures at which the desired product is not significantly decomposed while the liquid salt is being separated off and
c) the salt of the auxiliary base forms two immiscible liquid phases with the desired product or the solution of the desired product in a suitable solvent.

Preference is given to auxiliary bases which
a) do not participate as reactant in the reaction.

Also preferably, this auxiliary base can, in addition,
d) simultaneously function as nucleophilic catalyst in the reaction, i.e. it increases the reaction rate compared to a reaction carried out in the absence of an auxiliary base by a factor of at least 1.5, preferably by a factor of at least two, particularly preferably by a factor of five, very particularly preferably by a factor of at least ten and in particular by a factor of at least twenty.

The industrial usefulness of the method of the invention is that the auxiliary can be separated off by simple liquid-liquid phase separation at a low temperature, so that the handling of solids which is complicated from a process engineering point of view is dispensed with.

The work-up of the auxiliaries can also be carried out in the absence of the desired product, so the latter is stressed to a lesser extent.

The above-described object is achieved by the invention described here. This is brought about by the presence in or subsequent addition to reaction mixtures of auxiliary bases whose salts with acids which are eliminated during the course of the reaction or are added, i.e. are not eliminated during the reaction, are liquid under the reaction conditions and/or work-up conditions and form a phase which is immiscible with the optionally dissolved desired product. Such liquid salts are often referred to as ionic liquids. The acids to be bound can either be present in free form in the reaction mixture or form a complex or an adduct with the desired product or another substance which is present in the reaction mixture. Lewis acids in particular tend to form complexes with substances such as ketones. These complexes can be broken up by means of the auxiliary base to form, in the sense of the present invention, the salt of the auxiliary base and the Lewis acid to be separated off.

It is also possible to use mixtures or solutions of auxiliary bases in order to achieve the object of the invention.

For the purposes of the present text, immiscible means that at least two liquid phases separated by a phase boundary are formed.

If the pure desired product is completely or largely miscible with the salt of the auxiliary base and the acid, an auxiliary, e.g. a solvent, can also be added to the desired product to achieve demixing or a reduction in solubility. This is, for example, useful when the solubility of the salt in the desired product or vice versa is 20% by weight or more, preferably 15% by weight or more, particularly preferably 10% by weight or more and very particularly preferably 5% by weight or more. The solubility is determined under the conditions of the respective separation. The solubility is preferably determined at a temperature above the melting point of the salt and below the lowest of the following temperatures, particularly preferably 10° C. below the lowest and very particularly preferably 20° C. below the lowest:
boiling point of the desired product
boiling point of the solvent
temperature of significant decomposition of the desired product, depending on which temperature is the lowest.

The solvent can be considered to be advantageous when the mixture of desired product and solvent is able to dissolve the salt or the salt is able to dissolve the desired product or a mixture of desired product and solvent to a lesser extent than that indicated above.

Solvents which can be used are, for example, benzene, toluene, o-, m- or p-xylene, cyclohexane, cyclopentane, pentane, hexane, heptane, octane, petroleum ether, acetone, isobutyl methyl ketone, diethyl ketone, diethyl ether, tert-butyl methyl ether, tert-butyl ethyl ether, tetrahydrofuran, dioxane, ethyl acetate, methyl acetate, dimethylformamide, dimethyl sulfoxide, acetonitrile, chloroform, dichloromethane, methylchloroform or mixtures thereof.

The desired product is generally a nonpolar organic or inorganic compound.

Possible chemical reactions on which the invention is based are all reactions in which acids are liberated.

Reactions for which the method of the invention can be employed are, for example,
alkylations with alkyl or aralkyl halides, e.g. methyl chloride, methyl iodide, benzyl chloride, 1,2-dichloroethane or 2-chloroethanol,
acylations, i.e. reactions of acid halides and carboxylic anhydrides, of any substrates, for example alcohols or amines,
silylations, i.e. reactions with compounds containing at least one Si-halogen bond, e.g. SiCl₄, (H₃C)₂SiCl₂ or trimethylsilyl chloride,
phosphorylations, i.e. reaction with compounds containing at least one P-halogen bond, e.g. PCl₃, PCl₅, POCl₃, POBr₃, dichlorophenylphosphine or diphenylchlorophosphine, as are described, for example, by Julian Chojnowski, Marek Cypryk, Witold Fortuniak, Heteroatom. Chemistry, 1991, 2, 63-70,
sulfurations, e.g. sulfidations, introduction of —SO₃H, sulfonations and sulfations, by means of, for example, sulfuryl chloride (SO₂Cl₂), thionyl chloride (SOCl₂), chlorosulfonic acid (ClSO₃H), sulfonyl halides, e.g. p-toluenesulfonyl chloride, methanesulfonyl chloride or trifluoromethanesulfonyl chloride, or sulfonic anhydrides, as are described, for example, by Dobrynin, V. N. et al. Bioorg. Khim. 9(5), 1983, 706-10,
eliminations in which a C═C double bond is formed with elimination of an acid, for example HCl, HBr, acetic acid or para-toluenesulfonic acid, or
deprotonations in which an acidic hydrogen atom is abstracted by the auxiliary base.

Among the types of reaction mentioned, preference is given to alkylations, silylations, phosphorylations, sulfurations, acylations and eliminations and particular preference is given to silylations, phosphorylations and sulfurations.

Furthermore, the method of the invention can also be used for separating an acid from reaction mixtures to which an acid which has not been liberated during the reaction has been added, for example to adjust the pH or to catalyze a reaction. Thus, for example, Lewis acids which have been used as catalysts for Friedel-Crafts alkylations or acylations, can be separated off in a simple way.

The acids to be separated off according to the present invention can be Brönsted acids and Lewis acids. The definitions of Brönsted and Lewis acids are given in Hollemann-Wiberg, Lehrbuch der Anorganischen Chemie, 91st-100th edition, Walter de Gruyter, Berlin N.Y. 1985, p. 235 and p. 239, respectively. Lewis acids for the purposes of the present invention also include the Lewis acids used as Friedel-Crafts catalysts which are described in George A. Olah, Friedel-Crafts and Related Reactions, Vol. 1, 191 to 197, 201 and 284-90 (1963). Examples which may be mentioned are aluminum trichloride (AlCl₃), iron(III) chloride (FeCl₃), aluminum tribromide (AlBr₃) and zinc chloride (ZnCl₂).

The Lewis acids which can be separated off according to the invention generally contain cationic forms of metals of groups Ib, IIb, IIIa, IIIb, IVa, IVb, Va, Vb, VIb, VIIb and VII of the Periodic Table of the Elements and also of the rare earths, for example lanthanum, cerium, praseodymium, neodymium, samarium, europium, gadolinium, terbium, dysprosium, holmium, erbium, thulium, ytterbium or lutetium.

Particular mention may be made of zinc, cadmium, beryllium, boron, aluminum, gallium, indium, thallium, titanium, zirconium, hafnium, erbium, germanium, tin, vanadium, niobium, scandium, yttrium, chromium, molybdenum, tungsten, manganese, rhenium, palladium, thorium, iron, copper and cobalt. Preference is given to boron, zinc, cadmium, titanium, tin, iron, cobalt.

Possible counterions of the Lewis acid are F⁻, Cl⁻, ClO⁻, ClO₃⁻, ClO₄⁻, Br⁻, J⁻, JO₃⁻, CN⁻, OCN⁻, SCN⁻, NO₂⁻, NO₃⁻, HCO₃⁻, CO₃²⁻, S₂⁻, SH⁻, HSO₃⁻, SO₃²⁻, HSO₄⁻, SO₄²⁻, S₂O₂²⁻, S₂O₄²⁻, S₂O₅²⁻, S₂O₆²⁻, S₂O₇²⁻, S₂O₈²⁻, H₂PO₂⁻, H₂PO₄⁻, HPO₄²⁻, PO₄³⁻, P₂O₇⁴⁻, dithiocarbamate, salicylate, (OCₙH₂ₙ₊₁)⁻, (CₙH₂ₙ₋₁O₂)⁻, (CₙH₂ₙ₋₃O₂)⁻ and (Cₙ₊₁H₂ₙ₋₂O₄)²⁻, where n is an integer from 1 to 20, methanesulfonate (CH₃SO₃⁻), trifluoromethanesulfonate (CF₃SO₃⁻), toluenesulfonate (CH₃C₆H₄SO₃⁻), benzenesulfonate (C₆H₅SO₃⁻), hydroxide (OH⁻), anions of aromatic acids such as benzoic acid, phthalic acid and the like, and 1,3-dicarbonyl compounds.

Mention may also be made of carboxylates, in particular formate, acetate, trifluoroacetate, propionate, hexanoate and 2-ethylhexanoate, stearate and also oxalate, acetylacetonate, tartrate, acrylate and methacrylate, preferably formate, acetate, propionate, oxalate, acetylacetonate, acrylate and methacrylate.

Further possibilities are borohydrides and organoboron compounds of the general formulae BR″″₃ and B(OR″″)₃, where the radicals R″″ are each, independently of one another, hydrogen, C₁-C₁₈-alkyl, C₂-C₁₈-alkyl which may be interrupted by one or more oxygen and/or sulfur atoms and/or one or more substituted or unsubstituted imino groups, C₆-C₁₂-aryl, C₅-C₁₂-cycloalkyl or a five- to six-membered, oxygen-, nitrogen- and/or sulfur-containing heterocycle or two of them together form an unsaturated, saturated or aromatic ring which may be interrupted by one or more oxygen and/or sulfur atoms and/or one or more substituted or unsubstituted imino groups, where the radicals mentioned may each be substituted by functional groups, aryl, alkyl, aryloxy, alkyloxy, halogen, heteroatoms and/or heterocycles. The radicals R″″ may also be joined to one another.

In addition to the abovementioned AlCl₃, FeCl₃, AlBr₃ and ZnCl₂, preferred examples of Lewis acids are BeCl₂, ZnBr₂, ZnI₂, ZnSO₄, CuCl₂, CuCl, Cu(O₃SCF₃)₂, COCl₂, CoI₂, FeI₂, FeCl₂, FeCl₂(THF)₂, TiCl₄(THF)₂, TiCl₄, TiCl₃, ClTi(OiPr)₃, SnCl₂, SnCl₄, Sn(SO₄), Sn(SO₄)₂, MnCl₂, MnBr₂, ScCl₃, BPh₃, BCl₃, BBr₃, BF₃.OEt₂, BF₃.OMe₂, BF₃.MeOH, BF₃—CH₃COOH, BF₃.CH₃CN, B(CF₃COO)₃, B(OEt)₃, B(OMe)₃, B(OiPr)₃, PhB(OH)₂, 3-MeO-PhB(OH)₂, 4-MeO-PhB(OH)₂, 3-F-PhB(OH)₂, 4-F-PhB(OH)₂, (C₂H₅)₃Al, (C₂H₅)₂AlCl, (C₂H₅)AlCl₂, (C₈H₁₇)AlCl₂, (C₈H₁₇)₂AlCl, (iso-C₄H₉)₂AlCl, Ph₂AlCl, PhAlCl₂, Al(acac)₃, Al(OiPr)₃, Al(OnBu)₃, Al(OsecBu)₃, Al(OEt)₃, GaCl₃, ReCl₅, ZrCl₄, NbCl₅, VCl₃, CrCl₂, MoCl₅, YCl₃, CdCl₂, CdBr₂, SbCl₃, SbCl₅, BiCl₃, ZrCl₄, UCl₄, LaCl₃, CeCl₃, Er(O₃SCF₃), Yb(O₂CCF₃)₃, SmCl₃, Sml₂, B(C₆H₅)₃, TaCl₅.

The Lewis acids can be stabilized by alkali metal halides or alkaline earth metal halides, for example LiCl or NaCl. For this purpose, the alkali metal or alkaline earth metal halides are mixed with the Lewis acid in a molar ratio of 0-100:1.

For the purposes of the present text, halogen or Hal is fluorine (F), chlorine (Cl), bromine (Br) or iodine (I), preferably chlorine.

Compounds reacted in a silylation, phosphorylation or sulfuration are generally compounds which have at least one free O—H, S—H or N—H bond, if appropriate after deprotonation by the auxiliary base.

Acids which can form salts with the bases are, for example, hydroiodic acid (HI), hydrogen fluoride (HF), hydrogen chloride (HCl), nitric acid (HNO₃), nitrous acid (HNO₂), hydrobromic acid (HBr), carbonic acid (H₂CO₃), hydrogencarbonate (HCO₃—), methylcarbonic acid (HO(CO)OCH₃), ethylcarbonic acid (HO(CO)OC₂H₅), n-butylcarbonic acid, sulfuric acid (H₂SO₄), hydrogensulfate (HSO₄—), methylsulfuric acid (HO(SO₂)OCH₃), ethylsulfuric acid (HO(SO₂)OC₂H₅), phosphoric acid (H₃PO₄), dihydrogenphosphate (H₂PO₄⁻), formic acid (HCOOH), acetic acid (CH₃COOH), propionic acid, n-butyric and isobutyric acids, pivalic acid, para-toluenesulfonic acid, benzenesulfonic acid, benzoic acid, 2,4,6-trimethylbenzoic acid, mandelic acid, methanesulfonic acid, ethanesulfonic acid or trifluoromethanesulfonic acid, with preference being given to hydrogen chloride, acetic acid, p-toluenesulfonic acid, methanesulfonic acid, 2,4,6-trimethylbenzoic acid and trifluoromethanesulfonic acid and particular preference being given to hydrogen chloride.

In a preferred embodiment for separating off Brönsted acids (protic acids), these are separated off without large proportions of Lewis acids, i.e. the molar ratio of Brönsted acids to Lewis acids in the separated-off salt of the acid with the auxiliary base is greater than 4:1, preferably greater than 5:1, particularly preferably greater than 7:1, very particularly preferably greater than 9:1 and in particular greater than 20:1.

Preference is given to auxiliary bases whose salts of auxiliary bases and acids have a melting point at which no significant decomposition of the desired product, i.e. less than 10 mol % per hour, preferably less than 5 mol %/h, particularly preferably less than 2 mol %/h and very particularly preferably less than 1 mol %/h, occurs while the salt is being separated off as a liquid phase.

Among the abovementioned auxiliary bases, very particular preference is given to those whose salts have an ET(30) of >35, preferably >40, particularly preferably >42. The ET(30) is a measure of the polarity and is described by C. Reichardt in Reichardt, Christian Solvent Effects in Organic Chemistry Weinheim: VCH, 1979.-XI, (Monographs in Modern Chemistry; 3), ISBN 3-527-25793-4, page 241.

It is likewise possible to use all the abovementioned derivatives of imidazole whose salts have an ET(30) of >35, preferably >40, particularly preferably >42, and have a melting point at which no significant decomposition of the desired product occurs while the salt is being separated off as a liquid phase. The polar salts of these imidazoles form, as indicated above, two immiscible phases with less polar organic media.

The way in which the reaction is carried out is not subject to any restrictions and the reaction can, according to the invention, be carried out with neutralization of the liberated or added acids, if appropriate in the presence of nucleophilic catalysts, batchwise or continuously and in air or under a protective gas atmosphere.

In the case of temperature-sensitive desired products, it can be sufficient to allow the salt of auxiliary base and acid to precipitate as a solid salt during the reaction and only melt it for the work-up or after the major part of the desired product has been separated off in a solid-liquid separation. The product is subjected to less thermal stress as a result.

The invention further provides a method of separating the abovementioned auxiliary bases or auxiliary bases which are used as nucleophilic catalysts from a reaction mixture by admixing the reaction mixture with at least one mole of acid per mole of auxiliary base. This enables such auxiliary bases to be separated off as ionic liquids with the aid of a liquid-liquid separation.

The 1-alkylimidazoles can be recovered by, for example, liberating the salt of the auxiliary base by means of a strong base, e.g. NaOH, KOH, Ca(OH)₂, milk of lime, Na₂CO₃, NaHCO₃, K₂CO₃ or KHCO₃, if appropriate in a solvent such as water, methanol, ethanol, n-propanol or isopropanol, n-butanol, n-pentanol or butanol or pentanol isomer mixtures or acetone.

In a preferred embodiment of the invention, the strong base is used in a concentrated solution, particularly preferably an aqueous solution, for example a solution having a concentration of at least 5% by weight, preferably at least 10% by weight, and particularly preferably at least 15% by weight. This results in the reaction product of strong base and acid likewise being obtained in concentrated form and the liberated phase thus having a relatively low solubility in the other phase, i.e. preferably in the aqueous phase.

Further conceivable strong bases are amines, preferably tertiary amines, which are stronger bases, i.e. have a lower pK_{B}, than the 1-alkylimidazoles used according to the invention. Such amines can be, for example, trimethylamine, triethylamine, tri-n-butylamine, diisopropylethylamine, dimethylbenzylamine, pyridine, dimethylaminopyridine or strongly basic ion exchange resins. It would also be conceivable to use bases which are weaker than the 1-alkylimidazoles used according to the invention if the acid-base equilibrium established were able to be shifted by reaction engineering means, for example by removal of either the 1-alkylimidazole liberated or the salt of the weaker base by extraction, crystallization or distillation.

The auxiliary base which has been liberated in this way can, if it forms its own phase, be separated off, if it is miscible with the salt of the stronger base or the solution of the salt of the stronger base, be separated off from the mixture by distillation. If necessary, the auxiliary base liberated can also be separated from the salt of the stronger base or the solution of the salt of the stronger base by extraction with an extractant. Examples of extractants are the abovementioned solvents, alcohols or amines.

It is an advantage of the 1-alkylimidazoles used according to the invention over the prior art that the auxiliary bases liberated have only a low solubility in aqueous solutions and can thus be recovered with virtually no losses.

If necessary, the auxiliary base can be washed with water or aqueous NaCl or Na₂SO₄ solution and subsequently dried, e.g. by removal of any water present by means of an azeotropic distillation with benzene, toluene, xylene, butanol or cyclohexane.

If necessary, the base can be distilled before reuse.

The present invention further provides a method of separating acids from reaction mixtures by means of one of the abovementioned 1-alkylimidazoles, which comprises the following steps:

reacting at least one 1-alkylimidazole according to the invention with at least one acid in the presence of a desired product to form a mixture of at least one salt of the 1-alkylimidazole and the desired product,
separating the salt or salts of the 1-alkylimidazole and the desired product under conditions under which at least two separate phases of which at least one comprises pre-dominantly the salt or salts of the 1-alkylimidazole and at least one other comprises predominantly desired product are formed,
adding at least one base to a phase which has been separated off from (B) and comprises predominantly the salt or salts of the 1-alkylimidazole to form a mixture of the liberated 1-alkylimidazole and the reaction product of base and acid,
separating the mixture of the liberated 1-alkylimidazole and the reaction product of base and acid under conditions under which at least two separate phases of which at least one comprises predominantly the liberated 1-alkylimidazole in crude form and at least one other comprises the reaction product of base and acid are formed,
if appropriate, purifying the 1-alkylimidazole obtained in crude form and
if appropriate, recirculating the optionally purified 1-alkylimidazole to step (A).

The reaction of at least one of the 1-alkylimidazoles used according to the invention with at least one acid in the presence of a desired product in step (A) to form a mixture of at least one salt of the 1-alkylimidazole and the desired product has been described above. The acid can be, as described above, a Brönsted acid or a Lewis acid. The acid can be formed during a reaction, for example from the desired product being formed or as a coproduct, or can be added to the reaction mixture. According to the invention, pressure and temperature are not important in this step. It is likewise not critical whether the salt of the 1-alkylimidazole is or is not liquid in this step and whether desired product and the salt of the 1-alkylimidazole are miscible with one another or form separate phases at this stage.

The separation of the salt or salts of the 1-alkylimidazole and the desired product under conditions under which at least two separate phases of which at least one comprises predominantly the salt or salts of the 1-alkylimidazole and at least one other comprises predominantly desired product are formed is carried out in step (B). Here, the mixture from step (A) is brought to a temperature at which the salt of the 1-alkylimidazole is liquid and with the desired product forms, as described above, at least two immiscible phases.

As described above, at least one solvent can, if appropriate, be added to the reaction mixture in order to achieve demixing.

The separation is preferably carried out by phase separation (liquid-liquid separation), for example by means of techniques which are described in Ullmann's Encyclopedia of Industrial Chemistry, sixth edition, 2000 electronic release, chapter “Liquid-Liquid Extraction”, there in particular in subchapter 4 “Phase-Separation Equipment”, preferably by means of decantation, phase separators, centrifugation or mixer-settler apparatuses, and particularly preferably by means of phase separators.

In the present context “predominantly” means more than 50% by weight, preferably at least 66% by weight, particularly preferably at least 75% by weight, very particularly preferably at least 85% by weight and in particular at least 90% by weight, of the salt of the 1-alkylimidazole or desired product present in the total reaction mixture.

The desired product which has been separated off can subsequently be subjected to a purification known per se, which is not critical to the method of the invention.

In step (C), at least one base is added to a phase which has been separated off in (B) and comprises predominantly at least one salt of the 1-alkylimidazole to form a mixture of the liberated 1-alkylimidazole and the reaction product of base and acid.

As bases, it is possible to use the abovementioned strong bases, if appropriate in a solvent or with addition of a solvent, if necessary.

In an embodiment which is preferred according to the invention, the strong bases are used in aqueous solution. Since the 1-alkylimidazoles used according to the invention have a low solubility in aqueous solutions, at least two phases, viz. an aqueous phase which usually comprises the reaction product of base and acid and a phase comprising the 1-alkylimidazole liberated, are generally formed in step (C). This demixing process can, if necessary, be aided by addition of at least one solvent, but due to the low solubility of the 1-alkylimidazoles used according to the invention is usually and preferably not necessary.

The reaction product of base and acid is generally an aqueous solution of a salt, for example sodium, potassium or calcium chloride, bromide, acetate or formate.

As indicated above, the concentration of the strong base is preferably set so that the reaction product of acid and base is obtained in concentrated form, but preferably without precipitating under the separation conditions. The conditions are particularly preferably selected so that the reaction product of base and acid is obtained in an at least 15% strength by weight solution, very particularly preferably in an at least 20% strength by weight solution, in particular in an at least 25% strength by weight solution and especially in an at least 30% strength by weight solution.

The amount of base is usually selected according to the stoichiometry so that 0.8-1.5 equivalents, preferably from 0.9 to 1.3 equivalents, particularly preferably 0.95-1.2 equivalents and very particularly preferably 0.95-1.1 equivalents, of base are used, based on the amount of 1-alkylimidazole to be liberated. In particular, the base is used in an equimolar amount.

The temperature of the reaction is not critical to the invention; in general, warming has to be expected on addition of the base, so that slight cooling may be required. For example, the addition of the base can be carried out at a temperature of from 20 to 80° C.

In step (D), the mixture of the liberated 1-alkylimidazole and the reaction product of base and acid is separated under conditions under which at least two separate phases of which at least one comprises predominantly the liberated 1-alkylimidazole in crude form and at least one other comprises the reaction product of base and acid are formed.

Here, “predominantly” means more than 50% by weight, preferably at least 66% by weight, particularly preferably at least 75% by weight, very particularly preferably at least 85% by weight and in particular at least 90% by weight, of the 1-alkylimidazole or reaction product of acid and base present in the total mixture.

In this context, “crude” means having a purity of at least 75% by weight, preferably at least 85% by weight and particularly preferably at least 95% by weight, with solvents not being counted here.

The separation is usually and preferably a separation of two liquid phases which can generally be carried out as described under step (B). Should it be, in an exceptional case, a separation of a liquid from a solid, this can, for example, be carried out by single or multiple extraction or filtration, with the solid which remains being able to be washed with a solvent to remove adhering liquid.

The crude 1-alkylimidazole obtained from step (D) can optionally be purified in a further step (E). This can be carried out, for example, by single or multiple washing, drying, filtration, stripping, distillation and/or rectification.

To carry out washing, the 1-alkylimidazole is treated in at least one washing apparatus with water or a 5-30% strength by weight solution, preferably a 5-20% strength by weight solution, particularly preferably a 5-15% strength by weight solution, of sodium chloride, potassium chloride, ammonium chloride, sodium sulfate or ammonium sulfate, preferably of sodium chloride. Washing can be carried out, for example, in a stirred vessel or in other customary apparatuses, e.g. in a column or mixter-settler apparatus.

Drying can be achieved, for example, by removing any water present by means of a distillation or an azeotropic distillation with benzene, toluene, xylene, butanol or cyclohexane.

A filtration can be useful, for example, to remove precipitated solids or to eliminate a coloration which may occur, for example by filtration through activated carbon, aluminum oxide, Celite or silica gel.

A distillation, for example to separate off any solvent present, is preferably carried out in a falling film evaporator or thin film evaporator, if appropriate under reduced pressure, with a column being able to be superposed to improve separation.

The solvent can be reused in this form or, if appropriate, in purified form.

The worked-up and optionally purified 1-alkylimidazole can subsequently be returned to the process (step (F)).

The advantages of the present invention are that the selected 1-alkylimidazoles have a lower melting point than the auxiliary bases known from the prior art, for example from WO 03/62171, which means a reduced thermal stress on the desired product and a lower energy consumption, and in addition have a lower solubility, which leads to improved recoverability.

The following examples illustrate the invention without limiting its scope.

### EXAMPLES

In the present text “parts” or “%” are, unless indicated otherwise, “parts by weight” or “% by weight”.

### Preparation of the Imidazole Hydrochlorides and Determination of the Melting Point

The imidazole was dissolved in toluene and, while cooling in ice, treated with HCl gas until saturated. In general, either a solid precipitate or else an oil was formed immediately. Sometimes an only partly solid, partly oily product was obtained. In the first case, the solid precipitate was separated off directly by decantation and introduced into xylene. In the second case, the hydrochloride was dissolved completely by addition of ethanol and the solvent was subsequently removed completely under reduced pressure. Most hydrochlorides then crystallized after storage in a refrigerator.

To determine the melting point, xylene was added to the respective imidazole hydrochloride. On heating this heterogeneous mixture on an oil bath, melting of the lower phase was observed if the melting point was below 130° C. The internal temperature of the xylene was recorded as the melting point or melting range.

The results of these tests are shown in the table.

| | Substituent | Melting point [° C.] |
|---|---|---|
| Comparison | Me | 70 |
| Comparison | Et | 53 |
| | nPr | 38 |
| Comparison | iPr | 98 |
| | nBu | 29 |
| | iBu | 33 |
| Comparison | tBu | 76 |

### Determination of the Behavior of the 1-Alkylimidazoles Toward 30% Strength NaCl Solution

A solution of 30 g of sodium chloride in 100 g of demineralized water was prepared. 5 g of this solution were admixed with 5 g of the imidazole derivative listed in a shaking funnel and the mixture was shaken vigorously. The phases were then separated and weighed.

In the case of readily soluble imidazoles, part of the NaCl in the lower aqueous phase precipitated and was largely discharged with the lower phase. The weight of the upper phase was recorded.

For the purposes of analysis, a water determination was carried out on the upper phase (by Karl-Fischer titration). The lower phase was, if present, separated off, admixed with 1N KOH solution and extracted twice with xylene. After drying over magnesium sulfate, an internal standard (heptadecane) was added and the amount of dissolved imidazole was backcalculated after GC analysis.

| | Melting point | Solubility of free base | Solubility of |
|---|---|---|---|
| | (hydrochloride | in NaCl solution | water in imid- |
| Substituent | ° C.) | in % | azole in % |
| Me (comp.) | 70 | 100 | ca. 50 |
| Et (comp.) | 53 | 100 | ca. 50 |
| nPr | 38 | 3 | 30 |
| nBu | 29 | 0.2 | 16 |
| Hex | 50 | 0 | 8 |
| Oct | 55 | 0 | 6 |

### Preparation of Diethoxyphenylphosphine (Deopp) Using Butylimidazole

A solution of butylimidazole (26.1 g, 0.21 mol) in ethanol (9.44 g, 0.205 mol) was cooled in an ice bath and dichlorophenylphosphine (17.9 g, 0.10 mol) was added dropwise over a period of 30 minutes in such a way that the internal temperature did not exceed 40° C. The reaction mixture was then stirred for a further 30 minutes at this temperature and was subsequently transferred while warm to a separating funnel. After 30 minutes, the quite viscous lower phase was drained off and the upper phase was decanted. The lower phase was admixed with about 30 ml of toluene and mixed vigorously. Renewed phase separation while warm produced a toluene upper phase which was analyzed by gas chromatography using an internal standard (pentadecane). 16.8 g of NaOH solution (50% strength) and a little water (13.5 g) were then slowly added to the lower phase and the phases obtained were mixed vigorously. After renewed phase separation, the butylimidazole upper phase was analyzed by gas chromatography. The lower phase was extracted twice with xylene, the organic phases were dried and like-wise analyzed by gas chromatography using an internal standard (pentadecane).

1. A method of separating acids from reaction mixtures comprising the use of an auxiliary base, where the auxiliary base
A) reacts with the acid to form a salt which is liquid at temperatures at which the desired product is not significantly decomposed while the liquid salt is being separated off and
B) the salt of the auxiliary base forms two immiscible liquid phases with the desired product or the solution of the desired product in a suitable solvent,
and wherein the method the auxiliary base used is an alkylimidazole
which has a solubility in 30% strength by weight sodium chloride solution at 25° C. of 10% by weight or less and
whose hydrochloride has a melting point below 55° C.;
which comprises the following steps:
a) reacting at least one 1-alkylimidazole with at least one acid in the presence of a desired product to form a mixture of at least one salt of the 1-alkylimidazole and the desired product,
b) separating the salt or salts of the 1-alkylimidazole and the desired product under conditions under which at least two separate phases of which at least one comprises predominantly the salt or salts of the 1-alkylimidazole and at least one other comprises predominantly desired product are formed,
c) adding at least one base to a phase which has been separated off from (b) and comprises predominantly the salt or salts of the 1-alkylimidazole to form a mixture of the liberated 1-alkylimidazole and the reaction product of base and acid,
d) separating the mixture of the liberated 1-alkylimidazole and the reaction product of base and acid under conditions under which at least two separate phases of which at least one comprises predominantly the liberated 1-alkylimidazole in crude form and at least one other comprises the reaction product of base and acid are formed,
e) if appropriate, purifying the 1-alkylimidazole obtained in crude form and
f) if appropriate, recirculating the optionally purified 1-alkylimidazole to step (a).

2. The method according to claim 1, wherein a 1-alkylimidazole whose hydrochloride has a melting point below 45° C. is used.

3. The method according to claim 1, wherein a 1-alkylimidazole having a solubility in 30% strength by weight sodium chloride solution at 25° C. of 3% by weight or less is used.

4. The method according to claim 1, wherein the auxiliary base used is a 1-alkylimidazole of the formula (I), 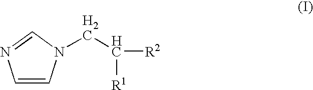
where R¹ and R² can each be, independently of one another, hydrogen or linear or branched C₁-C₆-alkyl, with the proviso that R¹ and R² have a total of at least 1 carbon atom and a total of not more than 6 carbon atoms.

5. The method according to claim 4, wherein R¹ and R² are selected independently from the group consisting of hydrogen, methyl and ethyl.

6. The method according to claim 1, wherein the 1-alkylimidazole is selected from the group consisting of 1-n-propylimidazole, 1-n-butylimidazole and 1-isobutylimidazole.

7. The method according to claim 6, wherein the separation of the phases in step (b) is carried out in a phase separator.

8. The method according to claim 1, wherein the concentration of the base or bases added in step (c) is selected so that the reaction product of base and acid in step (d) is obtained in at least 15% strength by weight solution.

9. The method according to claim 1, wherein the purification in step (e) comprises single or multiple washing, drying, filtration, stripping, distillation and/or rectification.

10. The method according to claim 2, wherein a 1-alkylimidazole having a solubility in 30% strength by weight sodium chloride solution at 25° C. of 3% by weight or less is used.

11. The method according to claim 2, wherein the auxiliary base used is a 1-alkylimidazole of the formula (I), 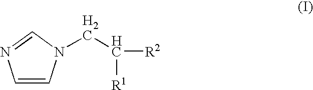
where R¹ and R² can each be, independently of one another, hydrogen or linear or branched C₁-C₆-alkyl, with the proviso that R¹ and R² have a total of at least 1 carbon atom and a total of not more than 6 carbon atoms.

12. The method according to claim 3, wherein the auxiliary base used is a 1-alkylimidazole of the formula (I), 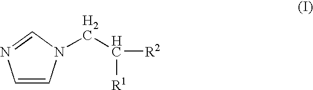
where R¹ and R² can each be, independently of one another, hydrogen or linear or branched C₁-C₆-alkyl, with the proviso that R¹ and R² have a total of at least 1 carbon atom and a total of not more than 6 carbon atoms.

13. The method according to claim 2, wherein the 1-alkylimidazole is selected from the group consisting of 1-n-propylimidazole, 1-n-butylimidazole and 1-isobutylimidazole.

14. The method according to claim 3, wherein the 1-alkylimidazole is selected from the group consisting of 1-n-propylimidazole, 1-n-butylimidazole and 1-isobutylimidazole.

15. The method according to claim 4, wherein the 1-alkylimidazole is selected from the group consisting of 1-n-propylimidazole, 1-n-butylimidazole and 1-isobutylimidazole.

16. The method according to claim 5, wherein the 1-alkylimidazole is selected from the group consisting of 1-n-propylimidazole, 1-n-butylimidazole and 1-isobutylimidazole.

17. The method according to claim 2, wherein the concentration of the base or bases added in step (c) is selected so that the reaction product of base and acid in step (d) is obtained in at least 15% strength by weight solution.

18. The method according to claim 3, wherein the concentration of the base or bases added in step (c) is selected so that the reaction product of base and acid in step (d) is obtained in at least 15% strength by weight solution.

19. The method according to claim 4, wherein the concentration of the base or bases added in step (c) is selected so that the reaction product of base and acid in step (d) is obtained in at least 15% strength by weight solution.

20. The method according to claim 5, wherein the concentration of the base or bases added in step (c) is selected so that the reaction product of base and acid in step (d) is obtained in at least 15% strength by weight solution.
